(19) United States
(12) Patent Application Publication
(10) Pub. No.: US 2009/0043123 A1
(43) Pub. Date: Feb. 12, 2009

(54) Dilute Stabilized Peracetic Acid Production and Treatment Process

(75) Inventors: William C. Copenhafer, Yardey, PA (US); Charles J. Nicholson, III, Warrington, PA (US); Henry A. Pfeffer, Mercerville, NJ (US)

Correspondence Address: PATENT ADMINISTRATOR;FMC CORPORATION, 1735 MARKET STREET, PHILADELPHIA, PA, 19103, US

(73) Assignee: FMC Corporation, Philadelphia, PA (US)

(21) Appl. No.: 12/186,757

(22) Filed: Aug. 6, 2008

Related U.S. Application Data
(60) Provisional application No. 60/964,260, filed on Aug. 10, 2007.

(51) Int. Cl. C07C 409/26

(52) U.S. Cl. 562/6

(57) ABSTRACT

A process for the rapid production and stabilization of dilute aqueous peracetic acid in which acetic anhydride and hydrogen peroxide are reacted in an aqueous medium with a stoichiometric excess of hydrogen peroxide and the aqueous medium containing the peracetic acid reaction product is adjusted, as necessary, to a pH of less than about 8 to provide a stabilized dilute peracetic acid solution. The dilute peracetic acid may be produced on site or in situ for treatment of an aqueous medium requiring disinfecting, biocidal, antimicrobial or bleaching treatment.

### CROSS REFERENCE TO RELATED APPLICATION

This application claims the benefit of U.S. Provisional Application No. 60/964,260, filed Aug. 10, 2007.

### FIELD OF THE INVENTION

The present invention relates to a process for the rapid production of dilute peracetic acid that is stabilized against decomposition and, more particularly, to the production of dilute stabilized peracetic acid from the reaction of acetic anhydride and hydrogen peroxide.

### BACKGROUND OF THE INVENTION

Peracetic acid, sometimes called peroxyacetic acid or PAA, is a well known chemical for its strong oxidizing potential. Peracetic acid has a molecular formula of C₂H₄O₃ or CH₃COOOH, a molecular mass of 76.05 g/mol, and a molecular structure as follows:

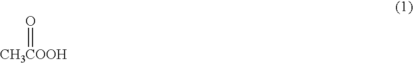

Peracetic acid is a liquid with an acrid odor and is normally sold in commercial formulations as aqueous solutions containing 5, 15 and 35 wt % peracetic acid. Such aqueous formulations typically contain peracetic acid as well as hydrogen peroxide (e.g., 7-25 wt %) and acetic acid (e.g., 6-39 wt %) in a dynamic chemical equilibrium.

Aqueous solutions of peracetic acid, at dilute concentrations below 5 wt % peracetic acid, are widely used in a variety of end use applications for their wide spectrum antimicrobial and biocidal properties, as a bactericide, fungicide, disinfectant and sterilant, and also for their bleaching properties. Peracetic acid exhibits antimicrobial activity that is more potent than hydrogen peroxide at equivalent low concentrations. A good overview of peracetic acid and its commercial antimicrobial applications is given by M. Kitis in “Disinfection of wastewater with peracetic acid: a review” Environment International 30 (2004) 47-55.

Peracetic acid is commonly manufactured by reaction of acetic acid and concentrated hydrogen peroxide, with an acid catalyst, e.g., sulfuric acid, in a continuous process that is allowed to proceed for days in order to achieve high reaction yields.

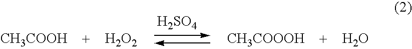

The reaction rate is proportional to the concentration of the reactants present, so preparation of less concentrated solutions of peracetic acid, e.g., less than about 10 wt % peracetic acid, typically requires uneconomically long reaction times.

As a result of these reaction kinetics, dilute concentrations of peracetic acid are normally prepared by water dilution of more concentrated peracetic acid solutions. However, dilution of concentrated peracetic acid solutions with water usually results in the hydrolysis of some of the peracetic acid and its decomposition into acetic acid, which reduces the amount of available peracetic acid.

An alternative method for the production of peracetic acetic has been described in the literature, involving the reaction of acetic anhydride (rather than acetic acid) and hydrogen peroxide. U.S. Pat. No. 2,377,038 of Reichert et al. teaches a method for making dilute peracid, including peracetic acid, solutions by reacting a peroxygen compound (e.g., alkali metal peroxide, hydrogen peroxide or sodium perborate) with an acid anhydride (acetic anhydride for peracetic acid), using equimolar (stoichiometric) amounts of the two reactants.

U.S. Pat. No. 3,432,546 of Oringer et al. teaches the production of peracetic acid using a tubular reactor that provides turbulent mixing of the acetic anhydride and hydrogen peroxide reactants, in the presence of an alkaline catalyst like sodium hydroxide. The process uses stoichiometric or slightly less than stoichiometric amounts of hydrogen peroxide in the reaction with acetic anhydride.

U.S. Pat. No. 5,977,403 of Byers teaches the production of peracetic acid in a two step process by reacting acetic anhydride with concentrated hydrogen peroxide and then diluting the resulting acid solution to obtain a dilute peracetic acid solution.

There remains a need for a direct, fast and cost effective method for producing dilute aqueous peracetic acid solutions, particularly at the site where such solutions are employed in various end use applications.

### BRIEF SUMMARY OF THE INVENTION

In accordance with the present invention, peracetic acid is prepared in a process for the rapid production and stabilization of dilute aqueous peracetic acid comprising introducing acetic anhydride into an aqueous medium in the presence of hydrogen peroxide, the aqueous medium being at a pH of about 5 to about 12 and having a mole ratio of hydrogen peroxide to acetic anhydride of greater than 1 to 1; providing sufficient amounts and mixing of the acetic anhydride and hydrogen peroxide in the aqueous medium to produce an aqueous peracetic acid reaction product having a concentration of at least about 50 ppm up to about 5 wt %; and adjusting the pH of the aqueous medium containing the peracetic acid reaction product, as necessary, to a pH of less than about 8 to provide a stabilized dilute peracetic acid reaction product.

Another embodiment of the present invention is a process for the rapid in situ production of dilute aqueous peracetic acid for treatment of an aqueous medium requiring treatment with an oxidizing agent comprising introducing acetic anhydride into a first aqueous medium in the presence of hydrogen peroxide, the first aqueous medium having a pH value of about 5 to about 12 and the relative amounts of hydrogen peroxide and acetic anhydride being adjusted to provide a mole ratio of hydrogen peroxide to acetic anhydride of greater than 1 to 1; providing sufficient mixing of the hydrogen peroxide and acetic anhydride to produce an aqueous peracetic acid reaction product in the first aqueous medium; contacting the first aqueous medium containing the aqueous peracetic acid reaction product with a second aqueous medium in need of treatment with an oxidizing agent, the amount of peracetic acid reaction product in the first aqueous medium being such to provide aqueous peracetic acid reaction product in the combined aqueous media having a concentration of at least about 1 ppm to about 5 wt %, to provide oxidizing activity in the combined aqueous media; and adjusting the pH of the combined first and second aqueous media, as necessary, to obtain a pH value of about 5 to about 8 to promote oxidizing activity of the peracetic acid reaction product in the combined media.

Still another embodiment of the present invention is a process for the rapid in situ production of dilute aqueous peracetic acid for treatment of an aqueous medium requiring treatment with an oxidizing agent comprising introducing hydrogen peroxide into an aqueous medium in need of treatment with an oxidizing agent, to provide a hydrogen peroxide concentration of less than about 5 wt % H₂O₂; adjusting the pH of the aqueous medium, as necessary, to obtain a pH value in the range of about 5 to about 12; introducing acetic anhydride into the aqueous medium in the presence of the hydrogen peroxide, to obtain a mole ratio of hydrogen peroxide to acetic anhydride in the aqueous medium of greater than 1 to 1; providing sufficient amounts and mixing of the acetic anhydride and hydrogen peroxide in the aqueous medium to produce a dilute aqueous peracetic acid reaction product in the aqueous reaction medium having a concentration of about 10 ppm up to about 1 wt %, available to provide oxidizing activity in the aqueous reaction medium; and adjusting the pH of the aqueous reaction medium, as necessary, to obtain a pH value of about 5 to about 8 to promote oxidizing activity of the peracetic acid reaction product in the aqueous reaction medium.

Yet another embodiment of the present invention is a process for the treatment of an aqueous medium requiring treatment with an oxidizing agent comprising treating an aqueous medium in need of treatment with an oxidizing agent with peracetic acid; adjusting the pH of the post-treatment aqueous medium to an alkaline pH value sufficient to decompose residual dilute peracetic acid in the treated aqueous stream; and thereafter readjusting the pH of the alkaline aqueous medium to a lower pH value suitable for discharge of the pH-readjusted aqueous stream into the environment.

### BRIEF DESCRIPTION OF THE DRAWINGS

FIG. 1 is a schematic flow diagram illustrating a preferred embodiment of the process of this invention, in which dilute peracetic acid is generated continuously in situ in an aqueous stream in need of treatment with an oxidizing agent such as dilute peracetic acid.

FIG. 2 is a schematic flow diagram illustrating another preferred embodiment of the process of this invention, in which dilute peracetic acid is generated continuously in situ in a sidestream diverted from an aqueous stream in need of treatment with an oxidizing agent and the peracetic acid-containing sidestream then returned to the aqueous stream in need of treatment.

FIG. 3 is a schematic flow diagram illustrating a third preferred embodiment of the process of this invention, in which dilute peracetic acid is generated on site in an aqueous medium which is then used to treat an aqueous stream in need of treatment with an oxidizing agent.

FIG. 4 is a graph showing the production concentration profiles of dilute peracetic acid in the process of this invention, as yield versus reaction time, at various pH values in the aqueous reaction medium for a fixed reactant ratio of hydrogen peroxide and acetic anhydride.

FIG. 5 is a graph showing the production concentration profiles of dilute peracetic acid in the process of this invention, as yield versus reaction time, at various reactant ratios of hydrogen peroxide and acetic anhydride in an aqueous medium at a fixed neutral pH.

### DETAILED DESCRIPTION OF THE INVENTION

The present invention is directed to the production of dilute peracetic acid in an aqueous medium, utilizing hydrogen peroxide and acetic anhydride as reactants. The process is particularly useful for the direct, on site production of peracetic acid, at the location where the peracetic acid is to be utilized for its intended application.

Hydrogen peroxide (H₂O₂) is a clear colorless liquid that is slightly more dense than water; hydrogen peroxide is a weak acid. Hydrogen peroxide is a strong oxidizer and decomposes exothermally into water and oxygen, making it a favored oxidizing agent. Hydrogen peroxide is miscible with water in all proportions and is available commercially at a wide range of concentrations, as concentrated aqueous solutions, e.g. 20, 35, 50 and 70 wt % aqueous H₂O₂, as well as more dilute aqueous solutions.

Acetic anhydride, also known as ethanoic anhydride or acetic acid anhydride and having the chemical formula (CH₃CO)₂O, is a widely available chemical reagent. Acetic anhydride is a colorless liquid with an acetic acid (vinegar) odor as a result of its reaction with moisture in air. Acetic anhydride is normally sold as the undiluted liquid, i.e., undiluted with water, since (i) its solubility in water is less than about 3 wt % and (ii) more importantly, it tends to hydrolyze in water to form acetic acid. Acetic anhydride is corrosive, an irritant, and flammable, so appropriate safety and handling measures must be employed in transport, storage and handling.

In the process of this invention, the reaction of hydrogen peroxide with acetic anhydride, in an aqueous medium, proceeds as follows, with equimolar amounts of hydrogen peroxide and acetic anhydride forming equimolar amounts of peracetic acid and acetic acid:

H₂O₂+(CH₃CO)₂O→CH₃COOOH+CH₃COOH (3)

In competition with this desired reaction is the tendency of acetic anhydride, in the presence of water, to hydrolyze to form acetic acid:

(CH₃CO)₂O+H₂O→2CH₃COOH (4)

This hydrolysis reaction is undesirable since it reduces the amount of acetic anhydride reactant otherwise available to react with hydrogen peroxide, according to reaction (3), leading to reduced reaction yields and reaction efficiencies.

The inventors have found, surprisingly, that acetic anhydride preferentially reacts with hydrogen peroxide that is present in an aqueous medium, rather than hydrolyzing with water, even at highly diluted hydrogen peroxide concentrations in the aqueous medium. For this reason, the order of addition of the hydrogen peroxide and acetic anhydride reactants is critical in the process of this present invention.

The peracetic acid process of this invention requires that the hydrogen peroxide reactant be present when the acetic anhydride reactant is introduced into the aqueous medium. Preferably, the aqueous medium already contains hydrogen peroxide, at a dilute concentration, available for reaction with the acetic anhydride that is introduced into the H₂O₂-containing aqueous medium. Alternatively, the hydrogen peroxide and acetic anhydride may be introduced into the aqueous medium concurrently or simultaneously, with thorough mixing being provided to ensure rapid contact of the acetic anhydride with the hydrogen peroxide, facilitating their reaction according to reaction (3).

The approach utilized in this invention increases the likelihood that the acetic anhydride will react preferentially with the hydrogen peroxide, to form the desired peracetic acid reaction product, rather than being decomposed by hydrolysis with water in the absence of hydrogen peroxide. The otherwise expected hydrolysis of acetic anhydride with water, to form unwanted acetic acid, does not occur to any significant extent since hydrogen peroxide, even at dilute or low concentrations, is present and available for reaction in the aqueous medium upon introduction of the acetic anhydride reactant.

In addition, since acetic anhydride has a tendency to hydrolyze with water, the present invention utilizes acetic anhydride in neat form, undiluted with water, as the reactant that is introduced into the aqueous medium in the presence of the hydrogen peroxide reactant.

The present invention thus avoids the situation where acetic anhydride is present or is introduced into the aqueous medium in advance of the hydrogen peroxide, where there is an increased likelihood that undesirable hydrolysis of the acetic anhydride is likely to occur (in the absence of the hydrogen peroxide reactant), resulting in the formation of acetic acid according to reaction (4).

The inventors have also found that the preferential reaction of acetic anhydride with hydrogen peroxide that is present in an aqueous medium (rather than acetic anhydride reacting with water) is enhanced in alkaline aqueous solutions, e.g., where the pH of the aqueous medium is above 8. The rate of formation of peracetic acid from the reaction of acetic anhydride with hydrogen peroxide is increasingly more rapid as pH of the aqueous medium becomes more alkaline.

Another feature of the process of this invention is the use of a stoichiometric excess of hydrogen peroxide relative to the acetic anhydride reactant, in the formation of peracetic acid according to reaction (3), and the use of relatively dilute concentrations of hydrogen peroxide in the aqueous medium.

Although the hydrogen peroxide reactant that is present in the aqueous solution is utilized in dilute concentrations, the process of this invention requires that the relative amount of hydrogen peroxide be in stoichiometric excess of the amount of acetic anhydride introduced into contact with the dilute hydrogen peroxide. The stoichiometric excess is greater than a 1:1 molar ratio of hydrogen peroxide to acetic anhydride. It should be noted that the reaction stoichiometry between hydrogen peroxide and acetic anhydride is equimolar, as shown in reaction (3) shown above.

In the process of this invention, the stoichiometric molar excess of hydrogen peroxide is preferably at least about 1.1:1, i.e., at least about [1.1 moles hydrogen peroxide]:[one mole acetic anhydride] and, more preferably, at least about 1.2:1 and, most preferably, at least about 1.5:1 moles hydrogen peroxide:mole acetic anhydride. Large stoichiometric molar excesses of hydrogen peroxide can also be used, e.g., up to about 6:1 moles hydrogen peroxide:mole acetic anhydride, or even higher, and the desired peracetic acid reaction product will still be obtained. Such large molar excesses of hydrogen peroxide are costly from an economic standpoint, so preferred molar excesses are typically maintained below about 3:1 moles hydrogen peroxide:mole acetic anhydride.

The stoichiometric excess of hydrogen peroxide used in the process of this invention serves several purposes. The excess of hydrogen peroxide ensures that there is a molar sufficiency of hydrogen peroxide available to react with the introduced acetic anhydride which is vulnerable to the competing hydrolysis reaction with water according to reaction (4). The stoichiometric excess of hydrogen peroxide ensures the availability of this reactant in its reaction with acetic anhydride, even in the event that some hydrogen peroxide reacts with or is decomposed by impurities in the aqueous medium. This is particularly important when the process of this invention is carried out in situ, using the aqueous medium being treated as the aqueous medium used to prepare the dilute peracetic acid solution. In addition, the excess of hydrogen peroxide serves to increase the driving force of the reaction rate between the hydrogen peroxide and acetic anhydride to make peracetic acid.

The use of a stoichiometric excess of hydrogen peroxide also reduces the likelihood that the acetic anhydride reactant will hydrolyze with water (a likely outcome in the absence of hydrogen peroxide), decomposing and forming acetic acid, which increases the biological oxygen demand (BOD) of the treated aqueous stream.

In the process of this invention, the hydrogen peroxide reactant is utilized as a relatively dilute aqueous solution, in its reaction in an aqueous medium with acetic anhydride to produce peracetic acid, the desired reaction product.

The dilute hydrogen peroxide in the aqueous medium may be present at a fairly wide range of concentrations, all of which are dilute. The hydrogen peroxide concentration in the aqueous medium may be up to 10 wt % H₂O₂ but is normally much less. The hydrogen peroxide concentration in the aqueous medium is preferably less than about 1 wt % H₂O₂ (10,000 ppm H₂O₂), and is more preferably less than about 0.5 wt % H₂O₂ (5,000 ppm H₂O₂), and is even more preferably less than about 0.1 wt % H₂O₂ (1,000 ppm H₂O₂).

The hydrogen peroxide concentration in the aqueous medium should be, at a minimum, at least about 0.01 wt % H₂O₂ (100 ppm H₂O₂).

The amount, or quantity, of hydrogen peroxide reactant present at these concentration levels in the aqueous medium must be sufficient to provide a stoichiometric excess of hydrogen peroxide, relative to the introduced acetic anhydride reactant.

The hydrogen peroxide source used to provide the dilute aqueous hydrogen peroxide employed in this invention is normally concentrated hydrogen peroxide. The hydrogen peroxide source may have a concentration in the range of from about 20 wt % H₂O₂ to about 70 wt % H₂O₂, but more dilute concentrations of hydrogen peroxide may also be used, e.g. about 5 up to about 20 wt % H₂O₂. The concentration of the hydrogen peroxide feed source used to prepare or provide the dilute hydrogen peroxide is not critical, since relatively dilute concentrations of hydrogen peroxide are ultimately and preferably employed in the aqueous medium of this invention (less than about 1 wt % H₂O₂) for reaction with the introduced acetic anhydride.

The hydrogen peroxide concentration of the feed source hydrogen peroxide is preferably in the range of about 5 wt % H₂O₂ to about 40 wt % H₂O₂. Commercial grades of hydrogen peroxide having concentrations of up to about 40 wt % H₂O₂ are preferred, since these concentrations are normally produced in commercial hydrogen peroxide production. Currently-offered commercial grades of hydrogen peroxide in excess of about 40 wt % H₂O₂ are generally more expensive (since they require additional process concentration steps such as distillation) and are therefore less preferred for the present invention, from an economic standpoint. Since concentrated hydrogen peroxide is classified as a strong oxidant and corrosive, appropriate transport, storage and handling precautions must be followed, in accordance with applicable material safety data sheets.

The hydrogen peroxide may alternatively be produced in situ in the aqueous medium from a hydrogen peroxide-generating source, e.g., a solid peroxygen compound that is a hydrogen peroxide source, introduced into the aqueous medium. Such hydrogen peroxide-generating solid compounds are characterized by their ability to generate the required hydrogen peroxide, as a decomposition product or the like, when introduced into or when dissolved or otherwise present in an aqueous medium.

The hydrogen peroxide-generating peroxygen compounds may be one or more solid peroxygen compounds. Examples include without limitation percarbonates like sodium percarbonate, perborates like sodium perborate, peroxides like sodium, magnesium or calcium peroxide, peroxyurea compounds, persilic acid, hydrogen peroxide adducts of pyrophosphates and phosphates like sodium phosphate perhydrate, and hydrogen peroxide adducts of citrates, sulfates, urea and sodium silicate, and the like, and mixtures thereof.

The process of the present invention utilizes several pH-related characteristics that the inventors have found provide unique advantages, including rapid production of dilute peracetic acid, stabilization of dilute peracetic acid solutions, and decomposition of residual peracetic acid in aqueous streams intended for discharge into the environment.

The present invention may be used to provide an enhanced reaction rate between acetic anhydride and dilute hydrogen peroxide to form peracetic acid in the process of this invention, by adjustment of the reaction pH of the aqueous reaction medium, as needed and desired, to an alkaline pH, e.g., above about 8 to about 12, that provides an enhanced reaction rate. The reaction may also be carried out at a neutral or slightly acidic pH although reaction rates will be somewhat slower.

The peracetic acid reaction product made in the process of this invention is stabilized by adjustment of the pH of aqueous reaction medium, following substantial completion of the reaction, by adjustment of the aqueous medium pH to a pH value below 8. The stabilization pH is preferably about 5 to less than 8, more preferably less than 7.5, and most preferably about 5.5 to about 7. In addition to providing enhanced stability, the stabilization pH below 8, particularly up to about pH 7, also enhances the oxidizing activity of the peracetic acid in various end-use antimicrobial or biocidal applications.

Specific desired pH values for the aqueous medium used for the reaction of the hydrogen peroxide and acetic anhydride reactants may be obtained or maintained by the introduction of a pH adjustment agent into the aqueous medium, prior to the production of the peracetic acid reaction product by reaction of acetic anhydride and hydrogen peroxide, to adjust the pH as necessary and as desired. In addition, such pH adjustment agents may also be used for pH adjustment after preparation of the peracetic acid reaction product, to reduce the pH to a value below about 8 or maintain the pH value at a specific pH value below about 8, or more preferably below about 7.5, to provide stabilization and enhanced oxidizing activity of the peracetic acid reaction product.

The pH adjustment agent may be selected from well known acidic and alkaline compounds typically used for pH adjustment of aqueous media to a specific pH value or pH value range.

For an acidic shift of the pH of the aqueous medium, the pH adjustment agent may be an acid or acidic compound, e.g., sulfuric acid, hydrochloric acid, nitric acid, phosphoric acid, citric acid, acetic acid, tartaric acid, succinic acid and other inorganic or organic acids, or acidic compounds, which are non-reactive with peracetic acid and mixtures thereof.

It should be recognized that the production of peracetic acid in the process of this invention, by reaction of hydrogen peroxide and acetic anhydride, in and of itself effects an acidic shift in the pH of the aqueous medium, with the formation of peracetic acid. This acidic shift in pH value of the aqueous medium during the peracetic acid formation can be exploited as one means for shifting the pH of aqueous solution to a more acidic pH value than that of the aqueous medium at the start of the reaction, thus enhancing the stability of the peracetic acid by shifting the aqueous medium pH below a pH value of 8.

For an alkaline shift of the pH of the aqueous medium, the pH adjusting agent may be an alkaline or basic compound or base, e.g., sodium hydroxide, calcium hydroxide, potassium hydroxide, sodium bicarbonate, sodium carbonate, any of the sodium phosphates, and other like inorganic or organic alkaline compounds and mixtures thereof.

The pH of the peracetic acid reaction product is normally maintained at a pH below about 8, in situations where enhanced stability of the peracetic acid is desirable or where enhanced activity of the peracetic acid is desirable in specific treatment end-use applications. In situations where the reaction is carried out at an alkaline pH, e.g., a pH in the range of about 8 to about 12, the pH of the resulting aqueous reaction product is normally adjusted below a pH value of 8, to provide a stabilized peracetic product.

The reaction between the hydrogen peroxide and acetic anhydride in the aqueous medium, to form dilute peracetic acid, increases in rate as the pH of the aqueous medium becomes more alkaline, generally providing higher yields of peracetic acid at shorter reaction times. This pH effect may be exploited in the in situ production of peracetic acid for immediate (in situ) treatment of an aqueous medium in need of treatment. Such rapid formation of peracetic acid is ideal for situations involving the combined in situ preparation of peracetic acid and the in situ treatment of the thus-formed peracetic acid. The long term stability of the peracetic acid is less important in such in situ treatments, since the peracetic acid is immediately available to provide its activity in the required treatment, e.g., antimicrobial or biocidal treatment. In such scenarios, stabilization of the peracetic acid thus formed is not critical and adjustment of aqueous medium pH below 8 is not absolutely essential.

In circumstances where the peracetic acid formed at alkaline pH values is required to be stored or held in a hold tank for any length of time, the highly alkaline pH values that facilitate the rapid production of peracetic acid may also lead to the subsequent decomposition of the peracetic acid reaction product. However, such decomposition can be avoided by adjustment of the pH of the aqueous medium to a pH below 8, i.e., a neutral or slightly acidic pH value.

The pH adjustment towards more acidic pH values may be carried out with any of the commonly-used mineral acids and similar acids. Sulfuric acid is preferred, but other acids such as those described earlier for acidic pH adjustment may also be used to lower the pH from an alkaline pH to a pH below 8.

Peracetic acid has an acid dissociation constant (pKₐ) value of 8.2, so peracetic acid in an aqueous solution at pH=8.2 is half dissociated, with about half being undissociated peracetic acid (protonated form) and half being the dissociated peracetate (deprotonated or salt form). In alkaline reaction conditions, e.g., above a pH value of about 8, it should be noted that the peracetic acid reaction product will be substantially in the dissociated form, a peracetic acid salt form or peracetate form. The term “peracetic acid reaction product” as used in this specification is intended to cover the undissociated peracetic acid form of the reaction product (typically present in neutral or slightly acidic aqueous solutions) and the dissociated peracetic acid salt or peracetate form (typically present in alkaline aqueous solutions, having a pH value of above about 8).

The process of the present invention calls for the pH of the aqueous peracetic acid reaction product to be at a pH value less than 8, to ensure that the predominant species of the peracetic acid reaction product is the undissociated acid form and to provide enhanced stability of the peracetic acid.

The final pH of the dilute aqueous peracetic acid reaction product solution is preferably in the range of about 5 to less than 8, more preferably less than about 7.5 and most preferably being in the range of about 5.5 to about 7. Such slightly acidic or neutral solutions of dilute aqueous peracetic acid, produced in the process of this invention, have been found to exhibit superior reaction efficacy and activity, when used for antimicrobial or biocidal end-use applications purposes, and also to provide good peracetic acid stability.

The peracetic acid product of this invention is relatively dilute, as compared with commercial grades of peracetic acid. The dilute peracetic acid made in the process of this invention may be produced in the aqueous medium over a wide range of concentrations, as low as 10 ppm peracetic acid and as high as up to about 5 wt % peracetic acid. The precise concentration of peracetic acid obtained in the process of this invention is controlled by suitable selection of the hydrogen peroxide and acetic anhydride reactant concentrations in the aqueous reaction medium, taking into account the molar ratio of hydrogen peroxide to acetic anhydride being used, as well as reaction yields, and like reaction parameters.

The stabilized dilute peracetic acid reaction product concentration produced in the aqueous medium by the preparation process of this invention is preferably at least 10 ppm, more preferably at least about 50 ppm and most preferably at least 100 ppm. The upper limit of the dilute peracetic acid reaction product may be as high as 5 wt %, but is preferably 3 wt % or less, more preferably 1 wt % or less, even more preferably 0.5 wt % (5000 ppm) or less and most preferably 0.3 wt % (3000 ppm) or less.

The concentration of dilute peracetic acid reaction product obtained in the process of this invention is typically controlled by adjustment of the amounts, e.g. concentrations, and mole ratio of the hydrogen peroxide and acetic anhydride reactants in the aqueous medium in which the reaction is carried out.

A distinction needs to be made between (i) the concentration of dilute peracetic acid reaction product produced in the aqueous reaction medium in the reaction between hydrogen peroxide and acetic anhydride (these peracetic acid reaction product concentrations having just been discussed above) and (ii) the concentration of dilute peracetic acid reaction product employed in the treatment of an aqueous stream or medium that requires or is otherwise in need of treatment with peracetic acid.

In treatment processes using dilute peracetic acid, the concentration of dilute peracetic acid reaction product can be as low as 1 ppm and still provide the desired activity, e.g., disinfecting, sanitizing, biocidal, antimicrobial or bleaching activity. Studies have shown that peracetic acid is very active even at very low concentrations, e.g., as low as 1 or 2 ppm. Low peracetic acid concentrations of about 5-10 ppm, for example, can provide disinfecting activity that accomplishes the desired disinfecting objective within minutes. Consequently, even though the minimum concentration of peracetic acid reaction product produced in the preparation process of this invention is very low, e.g. 10 ppm, the end use application could call for even lower concentrations that would be obtained by appropriate further dilution of the already dilute peracetic acid reaction product solution.

For industrial waste water treatment with dilute peracetic acid, the peracetic acid reaction product concentration is preferably less than about 3000 ppm. More preferably, the peracetic acid concentration is no more than about 2000 ppm, with concentrations in the range of about 300 ppm to about 1500 ppm also providing excellent antimicrobial or biocidal activity where such more concentrated peracetic acid solution treatments are desired. Lower concentrations of peracetic acid may also be used in various treatment situations, with the specific concentration or concentration range being dependent on the treatment requirements for the aqueous stream or medium in need of treatment.

For the in situ preparation of peracetic acid in the aqueous medium being treated, the relative amounts of hydrogen peroxide and acetic anhydride introduced into the aqueous medium are preferably adjusted to provide sufficient peracetic acid reaction product for the required disinfecting, antimicrobial, sterilizing or other treatment of the aqueous medium.

The process of this invention has the advantage of providing relatively rapid reaction times between the hydrogen peroxide and acetic anhydride reactants, to yield peracetic acid. The contact time between the acetic anhydride and hydrogen peroxide to produce the peracetic acid reaction product is normally less than about ten hours under most reaction conditions. As reaction conditions are optimized, e.g., though use of hydrogen peroxide to acetic anhydride molar ratios in excess of about 1.5:1, shorter contact times are possible, less than 6 or even 3 hours. Under optimal reaction conditions, the contact time between the hydrogen peroxide and acetic anhydride reactants can be an hour or less, even less than 30 minutes, with excellent reaction yields of peracetic acid product being obtained.

The rapid reaction times associated with the dilute peracetic acid production process of this invention provide several advantages over the prior art technique of diluting concentrated peracetic acid. Dilute peracetic acid is produced in the process of this invention either on an as-needed basis in relatively small amounts or in situ for direct treatment of the aqueous stream in need of disinfection, antimicrobial treatment or the like. Shipping and on site extended storage of concentrated or dilute peracetic acid are no longer required. Storage stability and corrosiveness of concentrated peracetic acid are not an issue. In addition, decomposition of peracetic acid to acetic acid during the dilution of concentrated peracetic acid is likewise not an issue, since the peracetic acid preparation process of this invention does not require a dilution step.

The temperature at which the peracetic acid reaction is carried out in the process of this invention is not critical. Temperatures of about 5° C. to about 80° C. are feasible, with temperatures in the range of about 10° C. to about 60° C. being preferred.

Yields of peracetic acid in the process of this invention are generally excellent, being at least about 50%, based on the amount of acetic anhydride reactant. Higher yields are more preferable, from an economic standpoint, and such yields are readily obtainable in the process of this invention. Preferably, the peracetic acid yield in the process of this invention is at least about 70%, based on the amount of acetic anhydride reactant employed, and, more preferably, at least about 80% and, most preferably, at least about 90%, all yields being based on the percentage of peracetic acid reaction product theoretically possible based on the amount of acetic anhydride reactant employed.

It should be recognized that the dilute peracetic acid is highly active as an oxidizing agent, disinfecting-sanitizing-biocidal-antimicrobial agent, or bleaching agent. Preparation of the dilute peracetic acid, in a preferred embodiment of this invention, in the aqueous medium or stream which is in need of such treatment will likely result in the peracetic acid reaction product quickly reacting further, to provide the desired treatment activity (e.g., oxidizing activity). This is particularly the case with the peracetic acid being used to treat organic pollutants or bacterial contaminants present in the aqueous medium used for in situ preparation of the peracetic acid. As a result, measurements of peracetic acid reaction product yields in such situations will be difficult to obtain with any degree of accuracy.

The process of the present invention for the production of dilute peracetic acid may be operated on a continuous, including semi-continuous, basis or as a batch wise operation.

Batch wise operation is favored where preparation of a quantity of dilute peracetic acid solution is desired in advance of the planned treatment procedure, the dilute peracetic acid solution being stored in a holding vessel or tank for use as needed. Continuous operation of the process of this invention is particularly useful for the in situ preparation of dilute peracetic acid, using the aqueous medium being treated with peracetic acid as the source of the aqueous medium in which the reaction of acetic anhydride with dilute hydrogen peroxide is carried out.

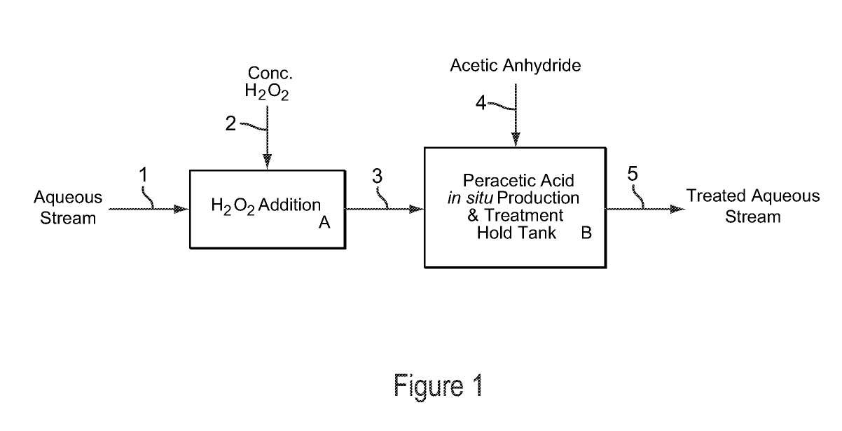

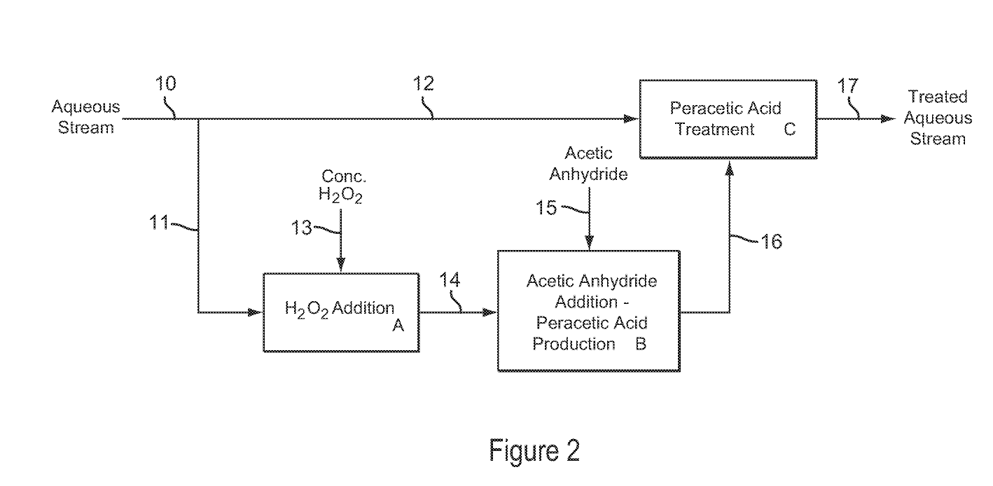

In one embodiment of continuous operation, dilute peracetic acid may be prepared on an as-needed basis, without maintenance of an “external” inventory. The dilute peracetic acid may be generated in situ, for direct treatment of an aqueous stream or medium in need of treatment. FIGS. 1 and 2 illustrate two embodiments of a continuously operated process, the first embodiment being a direct in situ generation of dilute peracetic acid in the aqueous medium in need of treatment and the second embodiment being a sidestream, in which dilute peracetic acid is generated, and then introduced into the aqueous medium in need of treatment. The sidestream variant may utilize a sidestream of the aqueous medium being treated as the source of the aqueous medium in which dilute peracetic acid is prepared. Alternatively, the sidestream may utilize water or other aqueous medium as the source of the aqueous medium in which the dilute peracetic acid is prepared.

For embodiments of the invention in which peracetic acid is prepared in situ, e.g., typically in a continuous operation for direct treatment of an aqueous medium, the reaction to form dilute peracetic acid must be relatively rapid. Such rapid generation of the dilute peracetic acid is required for successful treatment of the aqueous medium, since both generation of the peracetic acid and treatment of the aqueous medium in situ occur concurrently. For this reason, selection of an appropriate pH of the aqueous medium is a balancing act, on one hand to provide relatively rapid formation of peracetic acid, typically obtained with a pH of at least about 6, and on the other hand to provide optimal treatment time, e.g., disinfection of microorganisms, typically obtained with a pH less than about 8. The most preferred pH range for in situ continuous formation and treatment of an aqueous medium is therefore in the range of about 6 to less than 8.

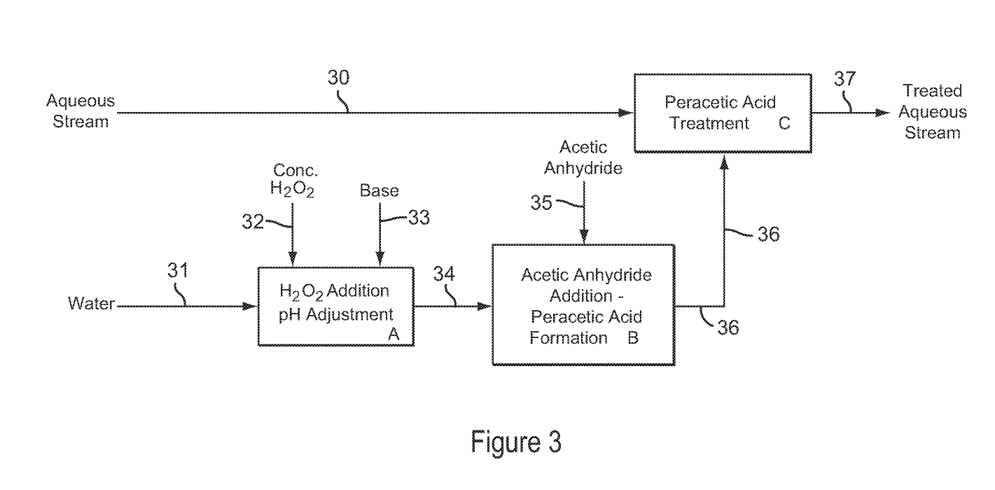

In a preferred embodiment of batch wise operation, small amounts of dilute peracetic acid may be prepared, e.g., using a simple tank reactor containing an aqueous medium into which the reactants are introduced to prepare dilute peracetic acid. The batch-prepared peracetic acid may then be metered continuously into the aqueous stream (or the like) in need of treatment. FIG. 3 depicts a schematic flow diagram of such batch wise peracetic acid preparation, where the batch-prepared inventory of relatively dilute peracetic acid is continuously introduced (metered) into an aqueous medium or stream in amounts sufficient to provide the needed disinfecting, sanitizing, biocidal or bleaching treatment.

For embodiments of the invention in which peracetic acid is prepared batch wise and stored, for subsequent treatment of the aqueous stream in need of treatment, it should be apparent that reaction rate during formation or preparation of the dilute peracetic acid is not critical, i.e., a very rapid reaction rate is not needed. In batch wise preparation of peracetic acid, the yield and stability of the peracetic acid reaction product are important objectives, so reaction parameters, including pH, are typically selected to optimize the yield and stability of the peracetic acid thus formed.

In any of the continuous, semi-continuous or batch wise operations, the process of this invention may be implemented without the need for specialized equipment and may be carried out at ambient temperatures and pressures. The process of this invention is very adaptable and enables the production of dilute peracetic acid in either relatively small amounts or in larger scale quantities. In addition, the flexibility of the process of the present invention permits dilute peracetic acid to be produced and stabilized at optimal pH values, while at the same time permitting subsequent pH adjustment of the treated stream, e.g., its neutralization to a pH value suitable for the treated stream's discharge into the environment.

An advantage of the present invention is the ability to prepare on site dilute aqueous solutions of peracetic acid, without the need for complex process manufacturing equipment, etc. The stabilized dilute aqueous peracetic acid solutions of this invention exhibit excellent stability, particularly short term stability of up to one week. Such peracetic acid stability ensures that most of the peracetic acid product formed will be available for antimicrobial or disinfecting treatments, with peracetic acid decomposition losses being minimized.

A primary utility for the stabilized dilute peracetic acid solution of the present invention is as antimicrobial or biocidal agents, including disinfecting, sanitizing and sterilizing end-use applications. The antimicrobial activity exhibited by these dilute aqueous peracetic acid solutions typically occurs within a short time of the peracetic acid being contacted with the solution in need of treatment. The antimicrobial activity of the dilute aqueous peracetic acid solutions is manifested within minutes, and can be substantially accomplished within about 1 minute to about 60 minutes, and more preferably within the range of 1 to about 30 minutes, of initial contact with the aqueous medium in need of treatment. Preferred contact times of the dilute aqueous peracetic acid with the aqueous medium in need of treatment are less than about 15 minutes and, more preferably, less than about 10 minutes. The actual time required will depend on factors such as the degree of mixing provided, pH and temperature of the aqueous medium being treated, the concentration of peracetic acid present, as well as the type and concentration of microbial components present in the aqueous medium.

These rapid treatment times make the process of this invention particularly well suited for the in situ treatment of aqueous streams or aqueous bodies in need of antimicrobial or biocidal treatment.

Dilute peracetic acid produced by the process of this invention has wide applicability as a disinfecting, sterilizing, biocidal or antimicrobial agent for the food processing, beverage, pharmaceutical and medical industries, industrial waste water, and as a bleaching agent in the textile, pulp and paper industries. The peracetic acid of this invention is especially useful for treating aqueous streams or aqueous media in such applications. Such water streams or media typically have pH values in the range of about 4 to about 8.

Dilute concentrations of peracetic acid exhibit broad-spectrum activity, with short contact times, against a wide range of microorganisms. The terms used in this specification that refer to end use applications for dilute peracetic acid have the following meanings. An “antimicrobial agent” is a substance that destroys or eliminates microbes, i.e., microorganisms, and a “biocidal agent” is a substance that kills organisms, usually in reference to microorganisms. A “bleaching agent” is a substance that whitens or decolorizes, e.g., textiles, pulp, paper or the like.

A “sanitizer” or “sanitizing agent” is a substance that significantly reduces the bacterial population in the inanimate environment, but does not destroy or eliminate all bacteria or other microorganisms. A “disinfectant” or “disinfecting agent” is a substance that destroys or eliminates a specific species of infectious or other public health microorganism, but not necessarily bacterial spores, in the inanimate environment. A “sterilant” or “sterilizing agent” is a substance that destroys or eliminates all forms of microbial life in the inanimate environment, including all forms of vegetative bacteria, bacterial spores, fungi, fungal spores, and viruses.

After treatment with the peracetic acid reaction product, whether an in situ treatment or otherwise, the aqueous medium in need of treatment with an oxidizing agent may be discharged into the environment, and this is preferably done after the pH of the aqueous medium is adjusted to a substantially neutral pH value, e.g., between about 6 to about 8, prior to such discharge.

There may be circumstances in which an aqueous medium is treated with peracetic acid but where residual peracetic acid remains present in the post-treatment aqueous medium. In such cases, it may be desirable to remove or decompose the remaining, residual amounts of peracetic acid, particularly if the aqueous medium is to be discharged into the environment, as a wastewater stream.

In such situations where there may be residual peracetic acid present in the treated aqueous solution, after the peracetic acid has accomplished its objective, e.g., decomposition, deactivation, neutralization (or the like) of unwanted elements originally present in the untreated aqueous stream. The treated stream may require further processing, in some circumstances, to remove the residual peracetic acid, before it can be discharged into the environment in accordance with applicable environmental or governmental regulations.

One approach for handling such residual peracetic acid, remaining from a process for the treatment of an aqueous medium in need of treatment with an oxidizing agent, involves treating an aqueous medium in need of treatment with an oxidizing agent with peracetic acid; adjusting the pH of the post-treatment aqueous medium to an alkaline pH value sufficient to decompose residual dilute peracetic acid in the treated aqueous stream; and thereafter readjusting the pH of the alkaline aqueous medium to a lower pH value suitable for discharge of the pH-readjusted aqueous steam into the environment.

The pH adjustment may be carried out using suitable pH adjustment agents, such as those described above, e.g., using sodium hydroxide for alkaline pH adjustment and sulfuric acid for acidic pH adjustment.

The pH adjustment of this process is preferably carried out such that the pH of the post-treatment aqueous medium is adjusted to a value above about 9 and, thereafter, the pH of the alkaline aqueous medium is readjusted to a value below about 9. The adjustment of the pH to an alkaline pH, e.g. a pH value of 9 or higher, serves to promote decomposition of the residual peracetic acid. A more preferred procedure uses a pH value of 10 or higher, rather than pH 9, in the peracetic acid decomposition procedure. The readjustment of the alkaline pH to a lower pH value, e.g., preferably below 9 and more preferably about 6 to 8, typically with an acidifying pH adjustment agent, permits the pH-readjusted aqueous steam to be safely discharged into the environment, in compliance with applicable regulations for such streams. The pH adjustment procedure for removing residual peracetic acid in a post-treatment aqueous stream may be coupled with a heat treatment as well, to promote decomposition of the residual peracetic acid, by raising the temperature of the aqueous stream containing the residual peracetic acid.

### EXAMPLES

The following non-limiting Examples illustrate preferred embodiments of the present invention.

### Example 1

In Example 1, an experimental study was carried out in laboratory-scale equipment to demonstrate the effect of reaction mixture pH on the formation of peracetic acid from the reaction of acetic anhydride and hydrogen peroxide in an aqueous medium.

The mole ratio of hydrogen peroxide to acetic anhydride used in this Example 1 was 5.7 moles H₂O₂ per mole of acetic anhydride, a mole ratio that provided a large stoichiometric excess of hydrogen peroxide. Three pH values were used: 10.0, 6.8 and 4.5. The aqueous medium in each study was appropriately buffered to maintain the specific pH value throughout the duration of the run.

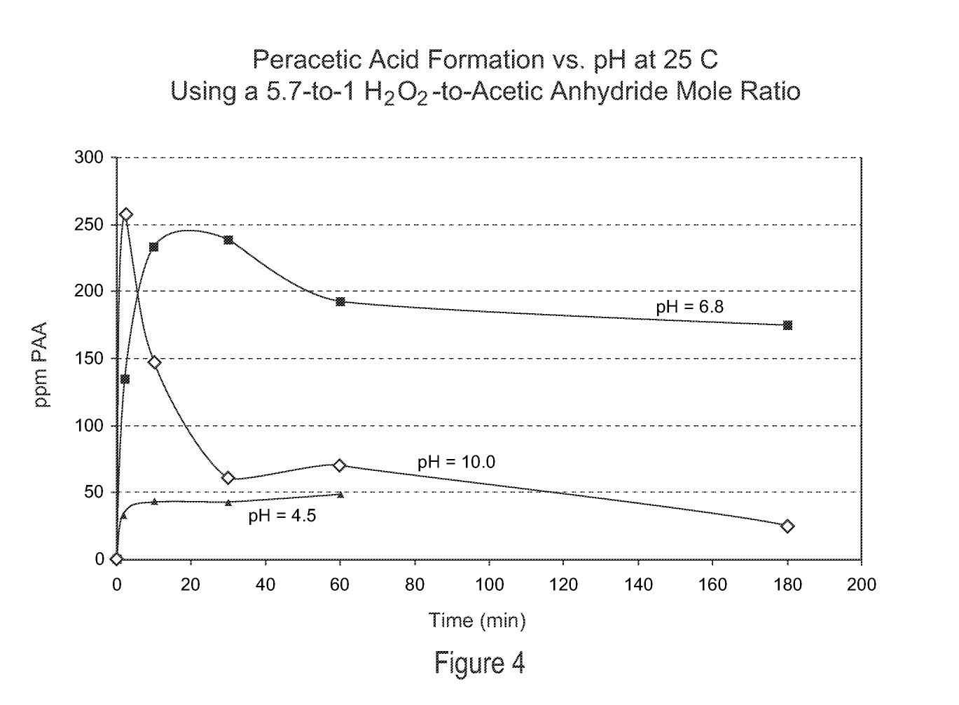

In each of the three runs, the operating procedure was as follows. A dilute aqueous buffered hydrogen peroxide solution, containing 585 ppm H₂O₂, was prepared and maintained at a temperature of 25° C. No hydrogen peroxide stabilizers were added to the solution. Acetic anhydride, undiluted (i.e., 100%) and in an amount sufficient to provide the desired 5.7:1 mole ratio of H₂O₂:acetic anhydride, was added to the buffered hydrogen peroxide solution. Measurements of peracetic acid and hydrogen peroxide concentrations were obtained from samples taken periodically over a period of 180 minutes. Results of these three studies, as peracetic acid concentration vs. time, are plotted in the graph shown as FIG. 4.

The results shown in FIG. 4 demonstrate that peracetic acid formation (reaction rate) increases with increasing solution pH but also that peracetic acid decomposition rate increases with increasing solution pH. At pH 4.5, the reaction rate and peracetic acid yield were unacceptably low. At pH 6.8, the reaction yielded a maximum concentration of peracetic acid within a relatively short time, about 20 minutes, and peracetic stability was relatively good.

As shown in FIG. 4, formation of the peracetic acid was extremely fast at pH 10.0, yielding a maximum concentration of peracetic acid in less than five minutes. At pH 10.0, however, the decomposition of the peracetic acid formed was also high after the peracetic acid formation was complete.

The results shown in FIG. 4 indicate that, after peracetic acid is formed at an alkaline pH, the pH of the solution should be adjusted at least to a neutral pH and, more preferably, to an acidic pH value to retard decomposition of the quickly-formed peracetic acid.

In addition, the results at pH 10.0 shown in FIG. 4 demonstrate that any residual peracetic acid remaining in an aqueous medium after the desired treatment with peracetic acid may be quickly decomposed, by raising the pH of the treated solution to an alkaline pH.

The procedure followed in this Example 1 involved addition of the acetic anhydride to the aqueous solution containing dilute hydrogen peroxide. The importance of the order of addition is shown in the following comparative study (not illustrated in FIG. 4) that was also carried out, which emphasizes the importance of introducing undiluted acetic anhydride into contact with dilute hydrogen peroxide. The procedure described above, for the production of peracetic acid at a solution pH of 6.7, was repeated but this time the acetic anhydride was introduced into an aqueous solution, prior to the addition of the hydrogen peroxide one hour later. The relative amounts and concentration of acetic anhydride and hydrogen peroxide were otherwise the same.

In this comparative study, the maximum concentration of peracetic acid obtained was only 40 ppm, a yield less than 20% of the peracetic acid concentration obtained in the run at pH 6.7 shown in FIG. 4. These results suggested that most of the acetic anhydride, when introduced first to an aqueous solution without hydrogen peroxide being present, is probably hydrolyzed with water to yield acetic acid; this acetic acid hydrolysis reaction product does not react in any appreciable amount with hydrogen peroxide at low concentrations of the former.

### Example 2

In Example 2, another experimental study was carried out in laboratory-scale equipment to demonstrate the effect of the mole ratio of the hydrogen peroxide and acetic anhydride reactants on the formation of peracetic acid in an aqueous medium maintained at a single pH value, 6.8.

Three mole ratios of hydrogen peroxide to acetic anhydride were used in this Example 2: 5.7, 2.0 and 1.2 moles H₂O₂ per mole of acetic anhydride, all of which provided a stoichiometric excess of hydrogen peroxide. The aqueous medium in each study was appropriately buffered with a mixture of Na₂HPO₄/NaH₂PO₄ to maintain the pH value at 6.8 for the duration of the run.

In each of the three runs, the operating procedure was similar to that of Example 1 and was as follows. A dilute aqueous buffered hydrogen peroxide solution, containing respectively 585 ppm, 205 ppm, or 123 ppm H₂O₂ for the three mole ratios (5.7:1, 2.0:1 or 1.2:1 H₂O₂:acetic anhydride) was prepared and maintained at a temperature of 25° C. No hydrogen peroxide stabilizers were added to the solution. Acetic anhydride, undiluted (i.e., 100%) and in an amount sufficient to provide the desired mole ratio (5.7:1, 2.0:1 or 1.2:1 H₂O₂ :acetic anhydride), was added to the buffered hydrogen peroxide solution.

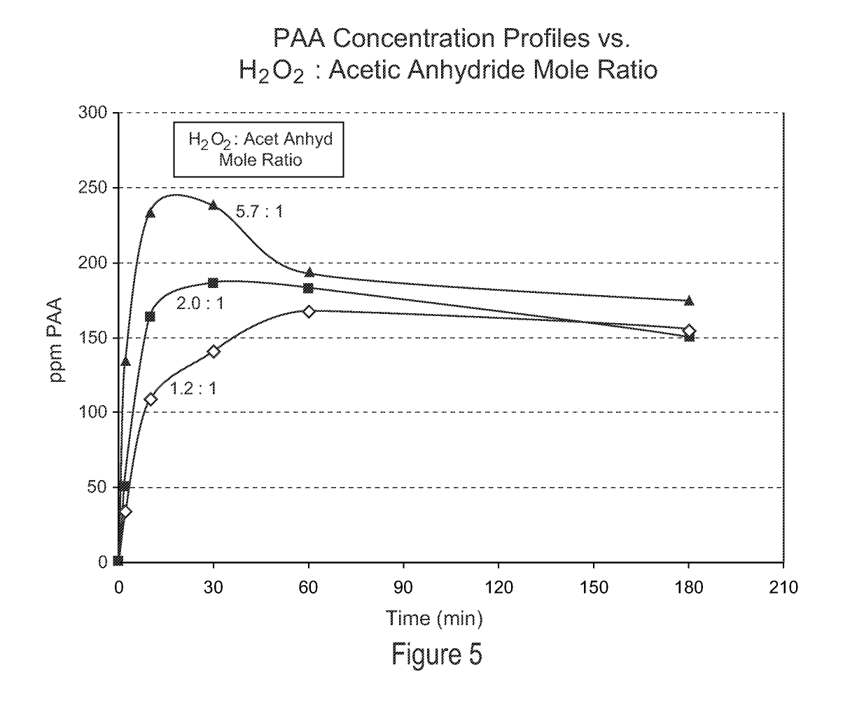

Measurements of peracetic acid and hydrogen peroxide concentrations were obtained from samples taken periodically over a period of 180 minutes. Results of these three studies, as peracetic acid concentration vs. time, are plotted in the graph shown as FIG. 5.

The results shown in FIG. 5 demonstrate that peracetic acid formation rate (reaction rate) increased as the mole ratio of hydrogen peroxide to acetic anhydride was increased. In addition, the maximum yield of peracetic acid obtained also increased with increasing mole ratios.

The reduced peracetic yield at lower mole ratios may be influenced by the decomposition of the thus-formed peracetic acid, with shorter reaction times (obtained at higher mole ratios) reducing the exposure of the peracetic acid (prior to maximum yield being obtained) to a competing decomposition reaction. For the highest mole ratio of 5.7:1, the maximum peracetic acid yield resulted after only about 20 minutes, but at the lowest mole ratio of 1.2:1, the maximum yield of peracetic acid was not obtained until 60 minutes had elapsed, allowing significant additional time for peracetic acid decomposition.

### Treatment Examples

The process of the present invention is especially useful for the on site production of dilute peracetic acid. Such on site production of peracetic acid is particularly advantageous for the immediate or in situ treatment of an aqueous stream that requires or is otherwise in need of an oxidizing treatment, for sanitizing, disinfecting, biocidal, antimicrobial, bleaching or other analogous purposes. The treatment processes illustrated in FIGS. 1, 2 and 3 show three alternative approaches, each involving the process of this invention to produce dilute peracetic acid that is used for treatment of an aqueous stream in need of treatment with an oxidizing agent. These treatment processes are described in Examples 3, 4 and 5, respectively.

### Example 3

Example 3 is a first preferred embodiment of the invention involving the production of dilute peracetic acid in the aqueous stream being treated, in an in situ production and treatment method, as is shown in the schematic flow diagram of FIG. 1. Referring now to FIG. 1, an aqueous waste water stream 1 from a food processing plant is treated with dilute peracetic acid that is produced directly in the aqueous stream, as follows. The aqueous waste water stream 1 is at a temperature of about 25° C. and has a pH value of about 7. The aqueous waste water stream 1 contains bacterial contaminants, and the stream 1 is treated with dilute peracetic acid, at a concentration of about 400-500 ppm, produced in situ for bactericidal treatment.

In FIG. 1, peracetic acid is produced in situ in the operations depicted by blocks A and B. Block A represents an inline mixer in which concentrated hydrogen peroxide 2, at 35 wt % H₂O₂, is metered into the aqueous stream 1 in an amount sufficient to yield a concentration of hydrogen peroxide of about 670 ppm H₂O₂ in the aqueous stream.

The H₂O₂-containing aqueous stream 3, at pH of 7, is then continuously passed to block B in FIG. 1, which represents a hold tank that provides a residence time of about 30 minutes. Acetic anhydride 4, undiluted with water, is metered into the aqueous stream 3 in the hold tank B an amount sufficient to provide a mole ratio of hydrogen peroxide to acetic anhydride in the aqueous stream of 3:1. Alternatively (and not shown in FIG. 1), the acetic anhydride 4 could be introduced into aqueous stream 3 upstream of the hold tank B (but downstream of the hydrogen peroxide introduction at block A) via an inline mixer and metering pump.

Hold tank B contains a mixing means to promote good contact of the stoichiometric excess of hydrogen peroxide and the introduced acetic anhydride reactants. The good mixing and stoichiometric excess of hydrogen peroxide result in the relatively rapid reaction and formation of dilute peracetic acid reaction product, within about 20 minutes after introduction of the acetic anhydride. Formation of the dilute peracetic acid causes the pH of the aqueous medium to become somewhat more acidic, with a pH value slightly below 7, which enhances the activity of the peracetic acid against the bacterial contaminants in the aqueous medium.

The peracetic acid formed in the aqueous stream in hold tank B reacts rapidly with the bacterial contaminants in the aqueous stream to decompose or otherwise inactivate them within a short time, less than about 5 minutes. The treated aqueous stream 5, which is continuously withdrawn from the holding tank B, has a substantially neutral pH and contains minimal residual peracetic acid. The treated aqueous stream 5 is suitable for discharge into the environment.

### Example 4

Example 4 is a second preferred embodiment of the treatment process of this invention and is illustrated in the schematic flow diagram shown in FIG. 2. This process embodiment involves the production of dilute peracetic acid in a sidestream 11 diverted from an aqueous stream 10 to be treated. The peracetic acid-containing sidestream 16 is then used to treat the main body 12 of the aqueous stream requiring treatment. The aqueous waste water stream 10 is at a temperature of about 25° C. and has a pH value of about 7.

As shown in FIG. 2, the aqueous waste water stream 10 from a food processing plant is treated with a sidestream 16 containing peracetic acid that is produced in situ in the sidestream, to provide a bactericidal treatment of bacterial contaminants present in the aqueous stream 10. Blocks A and B in FIG. 2 represent the operations carried out on the sidestream 11 to effect production of dilute peracetic acid. The peracetic acid-containing sidestream 16 contains about 1500-2000 ppm peracetic acid, and introduction of this sidestream 16 into the main aqueous stream 12 results in a peracetic acid treatment concentration of about 500 ppm in the combined streams 17.

Referring now to FIG. 2, a sidestream of about 20% of the stream 10 total volume flow rate is diverted as stream 11 for use in the production of peracetic acid in the sidestream itself. In FIG. 2, block A represents a mixing tank into which the aqueous sidestream 11 is introduced. Into this same tank A, concentrated hydrogen peroxide 13, at 35 wt % H₂O₂, is metered into tank A with mixing in an amount sufficient to yield a concentration of hydrogen peroxide of about 1610 ppm H₂O₂ in the aqueous medium.

Block B in FIG. 2 represents a second step that is carried out on the H₂O₂-containing aqueous medium 14. Block B can be a second tank but is preferably a second, subsequent step carried out in tank A. In this second step B, acetic anhydride 15, undiluted with water, is introduced with mixing into the aqueous H₂O₂-containing medium 14 in an amount sufficient to provide a mole ratio of hydrogen peroxide to acetic anhydride in the aqueous medium of 1.8:1.

The mixing that occurs in step B in holding tank A facilitates the rapid reaction of the stoichiometric excess of hydrogen peroxide present in the aqueous medium with the introduced acetic anhydride 15 to form dilute peracetic acid reaction product. The resulting peracetic acid reaction product is formed quickly, within about 20 minutes after introduction of the acetic anhydride. Formation of the dilute peracetic acid reaction product causes the pH of the aqueous reaction mixture to become slightly more acidic, with a pH value below 7.

In the final step of the process of this invention shown in FIG. 2, the pH-adjusted aqueous side stream 16 containing a stabilized peracetic acid solution is reintroduced into the main stream 12, to provide the desired peracetic acid bactericidal treatment. The side stream 16 from block B, containing peracetic acid reaction product, is continuously reintroduced into the main stream 12 in block C, thus effecting the desired peracetic acid treatment of the stream 12 and an effective peracetic acid concentration of about 500 ppm in the resulting aqueous stream 17.

The recombination of the side stream 16 and main stream 12 in the peracetic acid treatment of block C may be carried out in a holding tank that provides sufficient residence time for the bactericidal treatment. The peracetic acid in the peracetic acid treatment stream 17 provides immediate bactericidal activity required for rapid treatment of the aqueous stream 12, with the contact time (residence time in the holding tank) needed for treatment being less than about 10 minutes.

There is no appreciable residual concentration of unreacted peracetic acid in stream 17 following the peracetic acid treatment in the holding tank of block C. The treated stream 17 has a substantially neutral pH value of about 6-7 and does not require further pH adjustment prior to its discharge into the environment.

### Example 5

Example 5 is a third preferred embodiment of the invention involving the on site batch production of peracetic acid and use of this peracetic acid in a continuous treatment procedure, as is shown in the schematic flow diagram of FIG. 3. Referring now to FIG. 3, an aqueous waste water stream 30 containing bacterial contaminants, from a food processing plant, is subjected to peracetic acid treatment, shown as block C, using peracetic acid 36 that is produced at the treatment site. The aqueous waste water stream 30 being treated is at a temperature of about 25° C. and has a pH value of about 7.

Peracetic acid solution 36, having a peracetic acid concentration of about 1500-2000 ppm, is introduced in the peracetic acid treatment step C to in an amount sufficient to provide about 500 ppm peracetic acid in the treated aqueous stream 37.

The peracetic acid solution 36 used in the treatment step C is prepared in a batch wise procedure that is shown in FIG. 3. Water 31 is introduced to a stirred tank A, and concentrated hydrogen peroxide 32, at 35 wt % H₂O₂, is metered into tank A with mixing in an amount sufficient to yield a diluted hydrogen peroxide concentration of about 580 ppm H₂O₂. In addition, a base 33, which is 5 wt % aqueous NaOH, is likewise metered into tank A with mixing, via a pH controlled pump, in an amount sufficient to adjust the pH of the aqueous hydrogen peroxide solution in tank A to a pH value of about 8.5. The slightly alkaline solution pH of 8.5 is advantageous for promoting the rapid reaction of hydrogen peroxide with the subsequently-introduced acetic anhydride.

Alternatively (not shown in FIG. 3), the pH adjustment could be carried out concurrently with the acetic anhydride addition, again using a pH controller for metering in the aqueous NaOH. In such an alternative embodiment, the endpoint pH for the aqueous medium containing the peracetic acid reaction product would be about 7-7.5, since the formation of peracetic acid causes a slight acidic shift of the solution pH.

Block B in FIG. 3 represents a second step that is carried out on the pH-adjusted H₂O₂-containing solution 34. Block B in FIG. 3 can be a second tank but is preferably a second, subsequent step B carried out in tank A. In this second step B, acetic anhydride 35, undiluted with water, is introduced with mixing into the aqueous solution 34 in an amount sufficient to provide a mole ratio of hydrogen peroxide to acetic anhydride in the aqueous solution of 1.3:1.

Mixing of the introduced acetic anhydride 35 with the H₂O₂-containing aqueous solution 34 facilitates the rapid reaction of hydrogen peroxide with the acetic anhydride in the aqueous reaction mixture to form dilute peracetic acid reaction product, having a concentration of about 1500-2000 ppm, within about one hour after introduction of the acetic anhydride.

Formation of the dilute peracetic acid causes the pH of the aqueous reaction mixture to become more acidic, with a pH value of about 7 or less. An even lower pH, of about 4-6, can optionally be obtained by the addition of an acidifying pH adjustment agent, e.g., 10 wt % aqueous sulfuric acid (not shown in FIG. 3), and this step is useful if the dilute peracetic acid solution is likely to be stored and used over a period of days, rather than used within one day. Such lower pH values promote stabilization of the peracetic acid in the aqueous reaction mixture, as well as enhancing the oxidizing (e.g., biocidal) activity of the peracetic acid.

The dilute peracetic acid solution in tank A, following completion of step B, is continuously introduced as stream 36, via a metering pump, into aqueous waste water stream 30, in a holding tank shown as block C, for the peracetic acid treatment of stream 30. The peracetic acid stream 36, having a peracetic acid concentration of about 1500-2000 ppm, is metered into stream 30 in peracetic acid treatment block C in an amount that provides about 500 ppm peracetic acid in the combined streams 30 and 36, which are subjected to mixing in hold tank C.

The bactericidal activity of the peracetic acid in the combined streams 30 and 36 in hold tank C is high, requiring only about 10-15 minutes of contact time (residence time in hold tank C) to provide the desired bactericidal treatment.

The treated aqueous steam 37 withdrawn continuously from the peracetic acid treatment hold tank C is substantially free of residual peracetic acid and, having close to a neutral pH, may be discharged into the environment.

In the event that treated stream 37 contains significant residual peracetic acid, the stream 37 may optionally be subjected to a final pH readjustment step (not shown in FIG. 3) to decompose any residual peracetic acid still present in the treated stream 37. In the optional peracetic acid decomposition step (not shown in FIG. 3), the pH of the aqueous stream 37 is first adjusted to a pH above 9, by the addition of base (e.g., 20 wt % aqueous NaOH) to effect decomposition of the residual peracetic acid at an alkaline pH. This step is followed by a pH readjustment below 9, to a substantially neutral pH, via the addition of an acid (e.g. 10 wt % aqueous sulfuric acid) in an amount sufficient to provide the desired pH readjustment. The pH readjustment procedure is preferably carried out using inline mixing devices, with the base and acid being metered in sequentially via pH sensor-controlled metering pumps. The pH-adjusted aqueous stream, containing no residual peracetic acid, may then be discharged into the environment or recycled for reuse.

It will be appreciated by those skilled in the art that changes could be made to the embodiments described above without departing from the broad inventive concept thereof. It is understood, therefore, that this invention is not limited to the particular embodiments disclosed but is intended to cover modifications within the spirit and scope of the present invention as defined by the appended claims.

What is claimed is:

1. A process for the rapid production and stabilization of dilute aqueous peracetic acid comprising
introducing acetic anhydride into an aqueous medium in the presence of hydrogen peroxide, the aqueous medium being at a pH of about 5 to about 12 and having a mole ratio of hydrogen peroxide to acetic anhydride of greater than 1 to 1;
providing sufficient amounts and mixing of the acetic anhydride and hydrogen peroxide in the aqueous medium to produce an aqueous peracetic acid reaction product having a concentration of at least about 50 ppm up to about 5 wt %; and
adjusting the pH of the aqueous medium containing the peracetic acid reaction product, as necessary, to a pH of less than about 8 to provide a stabilized dilute peracetic acid reaction product.

2. The process of claim 1 wherein the acetic anhydride is not diluted with water prior to its introduction into the aqueous medium.

3. The process of claim 1 wherein the mole ratio of hydrogen peroxide to acetic anhydride is at least about 1.2 to 1 up to about 6 to 1.

4. The process of claim 1 wherein hydrogen peroxide is introduced into the aqueous medium concurrently with the introduction of the acetic anhydride.

5. The process of claim 1 wherein hydrogen peroxide is introduced into the aqueous medium prior to the introduction of the acetic anhydride.

6. The process of claim 5 wherein the concentration of hydrogen peroxide in the aqueous medium is not more than about 10 wt % H₂O₂.

7. The process of claim 5 wherein the concentration of hydrogen peroxide in the aqueous medium prior to the addition of the acetic anhydride is at least about 50 ppm H₂O₂.

8. The process of claim 1 which further comprises introducing a pH adjustment agent into the aqueous medium, prior to production of the peracetic acid reaction product, to adjust the pH of the aqueous medium to a specific pH value.

9. The process of claim 1 wherein the pH of the aqueous medium prior to production of the peracetic acid reaction product is greater than about 7 up to about 11.

10. The process of claim 1 wherein the contact time between the acetic anhydride and hydrogen peroxide to produce the peracetic acid reaction product is less than about ten hours.

11. A process for the rapid in situ production of dilute aqueous peracetic acid for treatment of an aqueous medium requiring treatment with an oxidizing agent comprising
introducing acetic anhydride into a first aqueous medium in the presence of hydrogen peroxide, the first aqueous medium having a pH value of about 5 to about 12 and the relative amounts of hydrogen peroxide and acetic anhydride being adjusted to provide a mole ratio of hydrogen peroxide to acetic anhydride of greater than 1 to 1;
providing sufficient mixing of the hydrogen peroxide and acetic anhydride to produce an aqueous peracetic acid reaction product in the first aqueous medium;
contacting the first aqueous medium containing the aqueous peracetic acid reaction product with a second aqueous medium in need of treatment with an oxidizing agent, the amount of peracetic acid reaction product in the first aqueous medium being such to provide aqueous peracetic acid reaction product in the combined aqueous media having a concentration of at least about 1 ppm to about 5 wt %, to provide oxidizing activity in the combined aqueous media; and
adjusting the pH of the combined first and second aqueous media, as necessary, to obtain a pH value of about 5 to about 8 to promote oxidizing activity of the peracetic acid reaction product in the combined media.

12. The process of claim 11 wherein hydrogen peroxide is introduced into the aqueous medium prior to the introduction of the acetic anhydride.

13. The process of claim 11 wherein the hydrogen peroxide concentration in the first aqueous medium is less than about 10 wt % H₂O₂.

14. The process of claim 11 wherein the relative amounts of hydrogen peroxide and acetic anhydride in the first aqueous medium are adjusted to provide a mole ratio of hydrogen peroxide to acetic anhydride of at least about 1.2 to 1 up to about 6 to 1.

15. The process of claim 11 which further comprises introducing a pH adjustment agent into the aqueous medium, prior to the introduction of the acetic anhydride, to adjust the pH of the aqueous medium to a specific pH value.

16. The process of claim 11 which further comprises adjusting the pH, as necessary, of the first aqueous medium containing the peracetic acid reaction product to a pH value of about 5 to about 8 to stabilize the peracetic acid reaction product, prior to contacting the first aqueous medium with the second aqueous medium.

17. The process of claim 11 wherein the concentration of peracetic acid reaction product in the combined aqueous media is at least about 1 ppm to about 5 wt %.

18. The process of claim 11 wherein the first aqueous medium comprises a portion of the second aqueous medium which is diverted as a side stream, into which hydrogen peroxide and acetic anhydride are introduced.

19. The process of claim 11 wherein the aqueous medium being treated with peracetic acid reaction product is in need of disinfecting, antimicrobial or sterilizing treatment.

20. The process of claim 19 wherein the amounts of hydrogen peroxide and acetic anhydride introduced into the first aqueous medium are adjusted to provide sufficient peracetic acid reaction product for the required disinfecting, antimicrobial or sterilizing treatment of the aqueous medium.

21. A process for the rapid in situ production of dilute aqueous peracetic acid for treatment of an aqueous medium requiring treatment with an oxidizing agent comprising
introducing hydrogen peroxide into an aqueous medium in need of treatment with an oxidizing agent, to provide a hydrogen peroxide concentration of less than about 5 wt % H₂O₂;
adjusting the pH of the aqueous medium, as necessary, to obtain a pH value in the range of about 5 to about 12;
introducing acetic anhydride into the aqueous medium in the presence of the hydrogen peroxide, to obtain a mole ratio of hydrogen peroxide to acetic anhydride in the aqueous medium of greater than 1 to 1;
providing sufficient amounts and mixing of the acetic anhydride and hydrogen peroxide in the aqueous medium to produce a dilute aqueous peracetic acid reaction product in the aqueous reaction medium having a concentration of about 10 ppm up to about 1 wt %, available to provide oxidizing activity in the aqueous reaction medium; and
adjusting the pH of the aqueous reaction medium, as necessary, to obtain a pH value of about 5 to about 8 to promote oxidizing activity of the peracetic acid reaction product in the aqueous reaction medium.

22. The process of claim 21 wherein hydrogen peroxide is introduced into the aqueous medium prior to the introduction of the acetic anhydride.

23. The process of claim 21 wherein the mole ratio of hydrogen peroxide to acetic anhydride in the aqueous medium is at least about 1.2 to 1 up to about 6 to 1.

24. The process of claim 21 which further comprises introducing a pH adjustment agent into the aqueous medium, prior to production of the peracetic acid reaction product to adjust the pH of the aqueous medium to a specific pH value.

25. The process of claim 21 wherein the pH of the aqueous medium is adjusted, as necessary, prior to production of the peracetic acid reaction product to obtain a pH value in the range of about 6 to about 10.

26. The process of claim 21 wherein the concentration of peracetic acid reaction product produced in the aqueous reaction medium is less than about 3000 ppm.

27. The process of claim 21 wherein the aqueous medium being treated with peracetic acid reaction product is in need of disinfecting, antimicrobial or sterilizing treatment.

28. The process of claim 27 wherein the amounts of hydrogen peroxide and acetic anhydride introduced into the aqueous medium are adjusted to provide sufficient peracetic acid reaction product for the required disinfecting, antimicrobial or sterilizing treatment of the aqueous medium.

29. A process for the treatment of an aqueous medium requiring treatment with an oxidizing agent comprising
treating an aqueous medium in need of treatment with an oxidizing agent with peracetic acid;
adjusting the pH of the post-treatment aqueous medium to an alkaline pH value sufficient to decompose residual dilute peracetic acid in the treated aqueous stream; and thereafter
readjusting the pH of the alkaline aqueous medium to a lower pH value suitable for discharge of the pH-readjusted aqueous stream into the environment.

30. The process of claim 29 wherein the pH of the post-treatment aqueous medium is adjusted to a value above about 9 and the pH of the alkaline aqueous medium is readjusted to a value below about 9.
